(19) United States
(12) Patent Application Publication
(10) Pub. No.: US 2025/0318799 A1
(43) Pub. Date: Oct. 16, 2025

(54) MEDICAL INFORMATION PROCESSING APPARATUS, MEDICAL DIAGNOSIS APPARATUS, AND MEDICAL INFORMATION PROCESSING METHOD

(71) Applicant: Canon Medical Systems Corporation, Otawara-shi (JP)

(72) Inventors: Daisuke TAKAHASHI, Yaita (JP); Risa NAKASHIMA, Utsunomiya (JP); Seiichirou NAGAI, Otawara (JP); Hayato KASAOKA, Nasushiobara (JP); Shun AOKI, Nasushiobara (JP); Naoko KURATOMI, Sakura (JP); Takayuki TOMISAKI, Nasushiobara (JP)

(73) Assignee: Canon Medical Systems Corporation, Otawara-shi (JP)

(21) Appl. No.: 19/096,855

(22) Filed: Apr. 1, 2025

(30) Foreign Application Priority Data
Apr. 11, 2024 (JP) 2024-063757
Feb. 12, 2025 (JP) 2025-020928

(51) Int. Cl. A61B 6/58; G09B 23/00; G16H 40/40; G16H 70/20

(52) U.S. Cl. CPC A61B 6/586; G09B 23/00; G16H 40/40; G16H 70/20

(57) ABSTRACT

A medical information processing apparatus includes processing circuitry. The processing circuitry is configured to: acquire a user input concerning an operation of a medical apparatus; input the acquired user input concerning the operation of the medical apparatus into a trained model, and acquire an output from the trained model, the trained model having learned to, upon accepting, as an input, a user input, output an answer to the accepted user input; determine a feasibility of the acquired output in a local apparatus; and cause a display to display information corresponding to the output in accordance with the feasibility.

### CROSS-REFERENCE TO RELATED APPLICATION(S)

This application is based upon and claims the benefit of priority from Japanese Patent Applications No. 2024-063757, filed Apr. 11, 2024; and No. 2025-020928, filed Feb. 12, 2025; the entire contents of all of which are incorporated herein by reference.

### FIELD

Embodiments described herein relate generally to a medical information processing apparatus, a medical diagnosis apparatus, and a medical information processing method.

### BACKGROUND

Conventionally, user support for operations of medical diagnosis apparatuses such as X-ray diagnosis apparatuses have been offered through provision of operation manuals. If the user does not know how to operate a medical diagnosis apparatus, the user reads a manual to search for a relevant part or a solution. However, such a user support method is inefficient, since it takes a long time to arrive at an operation desired by the user in the manual. Moreover, depending on the situation of the apparatus, a plurality of operations need to be performed in a suitable order. There are also cases where an answer cannot be arrived at even by reading the manual. Furthermore, it is difficult for the user to determine the suitable order.

### BRIEF DESCRIPTION OF THE DRAWINGS

FIG. 1 is a block diagram showing an example of a configuration of an X-ray diagnosis apparatus according to a first embodiment.

FIG. 2 is a diagram showing an example of an outer configuration of the X-ray diagnosis apparatus according to the first embodiment.

FIG. 3 is a flowchart illustrating a procedure for an operation assisting process by the X-ray diagnosis apparatus according to the first embodiment.

FIG. 4 is a diagram showing an example of a display screen displayed on a display in the operation assisting process according to the first embodiment.

FIG. 5 is a diagram showing an example of a state in which a question has been input on the display screen of FIG. 4.

FIG. 6 is a flowchart illustrating a procedure for a determination process by the X-ray diagnosis apparatus according to the first embodiment.

FIG. 7 is a diagram showing an example of a state in which answers are displayed on the display screen of FIG. 4.

FIG. 8 is a diagram showing an example of a state in which solutions are displayed on the display screen of FIG. 4.

FIG. 9 is a diagram showing an example of a display screen displayed on a display in an operation assisting process according to a modification.

### DETAILED DESCRIPTION

In general, according to one embodiment, a medical information processing apparatus includes processing circuitry. The processing circuitry is configured to: acquire a user input concerning an operation of a medical apparatus; input the acquired user input concerning the operation of the medical apparatus into a trained model, and acquire an output from the trained model, the trained model having learned to, upon accepting, as an input, a user input, output an answer to the accepted user input; determine a feasibility of the acquired output in a local apparatus; and cause a display to display information corresponding to the output in accordance with the feasibility.

Hereinafter, embodiments of a medical diagnosis apparatus including a medical information processing apparatus will be described in detail with reference to the accompanying drawings. In the description that follows, structural elements having substantially the same functions and configurations will be denoted by the same reference symbols, and a duplicate description of such elements will be given only where necessary.

The medical diagnosis apparatus is, for example, an X-ray diagnosis apparatus, an X-ray computed tomography (CT) apparatus, a magnetic resonance imaging (MRI) apparatus, a positron emission tomography (PET) apparatus, or the like. The X-ray diagnosis apparatus is, for example, an X-ray TV couch apparatus. The medical diagnosis apparatus may also be referred to as a “medical image diagnosis apparatus”. The medical diagnosis apparatus may be, for example, a single-modality apparatus such as an X-ray diagnosis apparatus, or a multi-modality apparatus such as a PET/CT apparatus or a single-photon emission computed tomography (SPECT)/CT apparatus. In the embodiments to be described below, a medical information processing apparatus mounted on an X-ray diagnosis apparatus will be described as an example; however, the medical information processing apparatus may be mounted on a medical diagnosis apparatus of another type.

### First Embodiment

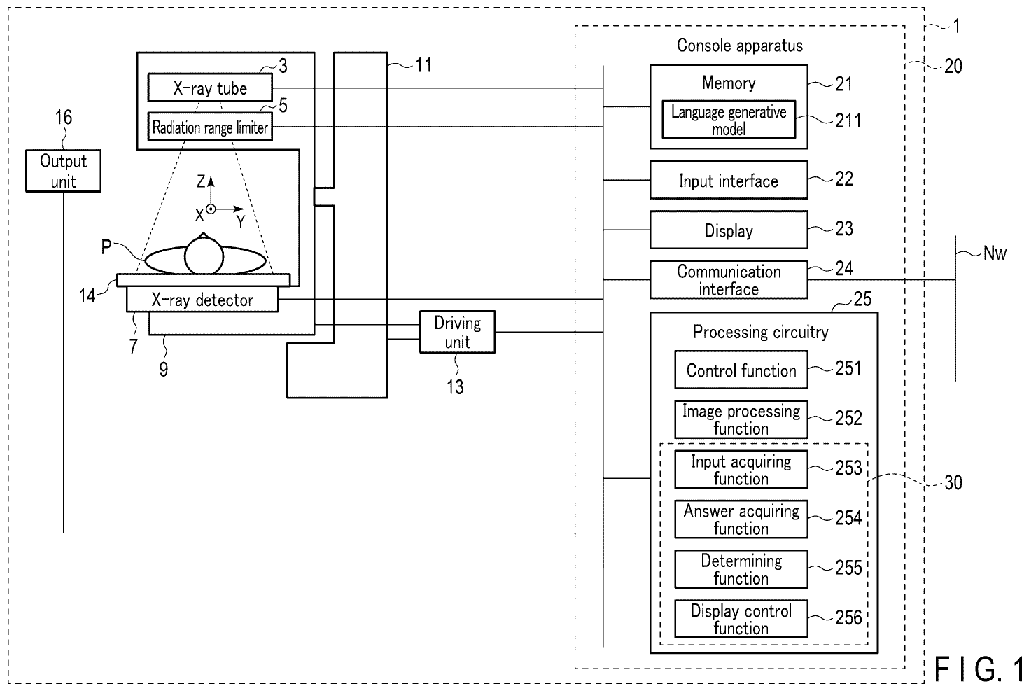

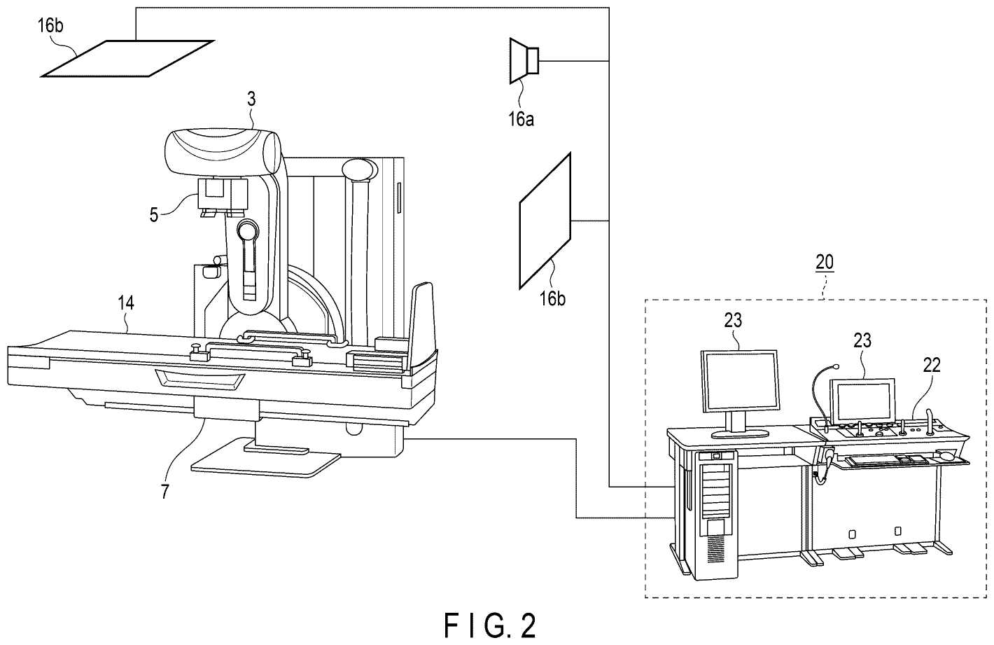

FIG. 1 is a block diagram showing an example of a configuration of an X-ray diagnosis apparatus 1 according to a first embodiment. FIG. 2 is a diagram showing an example of an outer configuration of the X-ray diagnosis apparatus 1 according to the first embodiment. The X-ray diagnosis apparatus 1 is a medical diagnosis apparatus including a medical information processing apparatus 30 according to the present embodiment. The X-ray diagnosis apparatus 1 according to the present embodiment is an X-ray TV couch apparatus used for gastrointestinal tract contrast examinations, etc.

As shown in FIGS. 1 and 2, the X-ray diagnosis apparatus 1 includes an X-ray tube 3, a radiation range limiter 5, an X-ray detector 7, a movement support mechanism 9, a rotation support mechanism 11, a driving unit 13, an output unit 16, and a console apparatus 20.

A fluoroscopy table includes a top plate 14 configured to support a subject; a movement support mechanism 9; an X-ray detector 7; and a rotation support mechanism 11. The fluoroscopy table is configured, for example, to raise and lower the top plate 14, and to move up and down, diagonally move, and compress an imaging system (the X-ray tube 3, the radiation range limiter 5, and the X-ray detector 7). The X-ray detector 7 is configured to detect X-rays that have passed through the subject and to transfer them to the console apparatus 20.

The X-ray tube 3 is connected to an unillustrated high-voltage generator. The high-voltage generator is configured to generate a tube current to be supplied to the X-ray tube 3 and a tube voltage to be applied to the X-ray tube 3. The high-voltage generator is configured to supply a tube current suitable for X-ray imaging and a tube current suitable for X-ray fluoroscopy to the X-ray tube 3, and applies a tube voltage suitable for X-ray imaging and a tube voltage suitable for X-ray fluoroscopy to the X-ray tube 3. Specifically, the high-voltage generator generates tube voltages and tube currents in accordance with X-ray imaging conditions under the control of processing circuitry 25 with a control function 251, to be discussed below.

The X-ray tube 3 is configured to generate X-rays from an X-ray focal point (hereinafter referred to as a “tube focal point”) based on the tube currents supplied from the high-voltage generator and the tube voltages applied by the high-voltage generator. The generated X-rays are emitted from an X-ray emission window of the X-ray tube 3. In the case of, for example, a gastrointestinal tract contrast examination, the X-ray tube 3 applies X-rays to a subject P into which a contrast agent and a blowing agent have been administered.

The radiation range limiter 5 is provided in front of the X-ray tube 3 and between the X-ray tube 3 and the X-ray detector 7. Specifically, the radiation range limiter 5 is provided in front of the X-ray emission window of the X-ray tube 3. The radiation range limiter 5 may also be referred to as an “X-ray adjustable diaphragm”. The radiation range limiter 5 is configured to limit the radiation range of the X-rays in such a manner that a portion other than a portion desired to be imaged by a user is not irradiated with the X-rays generated by the X-ray tube 3. The radiation range limiter 5 is configured, for example, to limit the radiation range by moving diaphragm blades in accordance with an instruction input by the input interface 22 to limit the radiation range.

The radiation range limiter 5 includes a plurality of diaphragm blades. Each of the diaphragm blades is configured of lead, which shields the X-rays generated by the X-ray tube 3. It is to be noted that the radiation range limiter 5 may further include a plurality of filters (hereinafter referred to as “additional filters”) to be inserted into the X-ray radiation field for the purpose of reducing the radiation dose to the subject P and improving the image quality. The additional filters may also be referred to as “X-ray filters”, “filtration plates”, “beam filters”, “radiation quality filters”, or “beam spectrogram filters”.

The X-ray detector 7 is configured to detect X-rays generated from the X-ray tube 3 that have passed through the subject P. The X-ray detector 7 is, for example, a flat panel detector (FPD). The X-ray detector 7 includes a plurality of semiconductor detection elements. The semiconductor detection elements are configured to convert incident X-rays into electric signals either by direct conversion or indirect conversion. In direct conversion, the incident X-rays are directly converted into electric signals. In indirect conversion, the incident X-rays are converted into light with a fluorescent material, and the light is converted into electric signals.

In accordance with the incident X-rays, the electric signals generated by the plurality of semiconductor detection elements are output to an unillustrated analog-to-digital converter (hereinafter referred to as an “A/D converter”). The A/D converter is configured to convert the electric signals into digital data. The A/D converter is configured to output the digital data into an unillustrated pre-processor. It is to be noted that, as the X-ray detector 7, an image intensifier, etc. may be used.

The movement support mechanism 9 is configured to support the imaging system (the X-ray tube 3, the radiation range limiter 5, and the X-ray detector 7) to allow its movement along a longitudinal-axis (an X-axis) of the top plate 14, under the control of the control function 251, to be discussed below. In the case where, for example, an imaging method such as long-length imaging in which a subject is imaged while moving the imaging system is input via the input interface 22, the movement support mechanism 9 moves the imaging system along a first direction in accordance with an imaging timing based on the input imaging method. It is to be noted that, if the imaging system does not need to be moved, the movement support mechanism 9 fixes the imaging system relative to the top plate 14, without moving it.

The rotation support mechanism 11 is configured to support the movement support mechanism 9 and the top plate 14 to allow them to rotate (be raised and lowered) around a short axis (a Y-axis) of the top plate 14 as a rotation axis. The movement support mechanism 9 includes the X-ray tube 3, the radiation range limiter 5, and the X-ray detector 7. The rotation support mechanism 11 is configured, for example, to cause the imaging system (the X-ray tube 3, the radiation range limiter 5, and the X-ray detector 7) or the top plate 14 to rotate around the rotation axis in response to the user's instruction via the input interface 22. The angle of rotation of the top plate 14 around the rotation axis is defined in such a manner, for example, that a rotation angle of the top plate 14 located at a horizontal position is 0°, and a rotation angle of the top plate 14 rotated to a position parallel to the vertical direction is 90°. It is to be noted that, in a state in which the top plate 14 is located at the horizontal position, a longitudinal direction of the top plate 14 becomes parallel to the X-axis, a short-side direction of the top plate 14 becomes parallel to the Y-axis, and a Z-axis orthogonal to the X-axis and the Y-axis become parallel to the vertical direction. Hereinafter, a position of the top plate 14 at the rotation angle of 90° will be referred to as “upright”. The subject P supported by the top plate 14 at the rotation angle of 90° is in an upright position.

It is to be noted that the movement support mechanism 9 and the rotation support mechanism 11 may be configured to support the X-ray tube 3, the radiation range limiter 5, the X-ray detector 7, and the top plate 14 along the orthogonal three axes (the X-axis, the Y-axis, and the Z-axis) shown in FIG. 1. For example, the movement support mechanism 9 is configured to support the X-ray tube 3, the radiation range limiter 5, and the X-ray detector 7 in such a manner that a distance between the X-ray generation focal point and the X-ray detector 7 (a source-to-image distance (hereinafter referred to as “SID”)) is variable.

The driving unit 13 is configured to drive the movement support mechanism 9 and the rotation support mechanism 11 under the control of the control function 251. Specifically, the driving unit 13 is configured to drive the rotation support mechanism 11 in accordance with a control signal from the control function 251, and rotates the rotation support mechanism 11 around the rotation axis. Thereby, constituent elements such as the top plate 14 are rotated around the rotation axis. Upon receiving, as an input, an instruction to arrange the top plate 14 in an upright position via the input interface 22, the driving unit 13 rotates the rotation support mechanism 11 to make the rotation angle of the top plate 14 90°. Also, the driving unit 13 is configured to move the imaging system along the X-axis direction by driving the movement support mechanism 9 in accordance with the user's instruction via the input interface 22.

An unillustrated top plate driving unit is configured to move the top plate 14 by driving the top plate 14 under the control of the control function 251. Specifically, the top plate driving unit is configured to slide the top plate 14 along the X-axis and Y-axis directions based on a control signal from the control function 251.

The output unit 16 includes a speaker 16a and a display 16b. The output unit 16 is controlled by the console apparatus 20, and is configured to output an instruction to the subject by speech or display. It is to be noted that one of the speaker 16a and the display 16b may be omitted.

The console apparatus 20 includes a memory 21, an input interface 22, a display 23, a communication interface 24, and processing circuitry 25. Data communications among the memory 21, the input interface 22, the display 23, the communication interface 24, and the processing circuitry 25 are performed by a BUS.

Hereinafter, the console apparatus 20 will be described as an apparatus configured to implement a plurality of functions with a single console; however, a plurality of functions may be implemented with separate consoles. For example, the functions of the processing circuitry 25, to be discussed below, may be mounted on different console apparatuses in a distributed manner.

The memory 21 is a storage device such as a read-only memory (ROM), a random access memory (RAM), a hard disk drive (HDD), a solid-state drive (SSD), or an integrated circuit storage unit, etc., configured to store various kinds of information. The memory 21 may be also a drive device, etc. configured to read and write a variety of information to and from a portable storage medium such as a CD-ROM drive, a DVD drive, a flash memory, or the like. It is to be noted that the memory 21 is not necessarily realized by a single storage device. For example, the memory 21 may be realized by a plurality of storage devices. Also, the memory 21 may be provided in another computer connected to the X-ray diagnosis apparatus 1 via a network.

The memory 21 is configured to store programs to be run by the processing circuitry 25 and various types of data to be used for processing in the processing circuitry 25. The various types of data are, for example, current examination images (a radiographic image and a fluoroscopic image obtained in a current examination) and past examination images (a radiographic image and a fluoroscopic image obtained in a past examination). Hereinafter, the radiographic images and the fluoroscopic images will be collectively referred to as “examination images” or “X-ray images”. The past examination images are acquired from an image server via a network by the processing circuitry 25, and are stored in the memory 21. As the programs, programs that can be installed onto a computer from a network or a non-transitory computer readable storage medium and that cause the computer to realize the functions of the processing circuitry 25 are used. Also, such programs may be stored and distributed in a non-transitory computer-readable storage medium, read from the non-transitory computer-readable storage medium, and installed in the memory 21. It is to be noted that various types of data handled herein are typically digital data. The memory 21 is an example of a storage unit.

The memory 21 is configured to store a language generative model 211. The language generative model 211 is a trained model having trained to, upon accepting, as an input, a question concerning an operation of a medical apparatus, output an answer to the question. As the language generative model 211, a language generative artificial intelligence (AI) such as ChatGPT (registered trademark) may be used; however, language generative AI other than ChatGPT or a trained model other than the language generative AI may also be used. The language generative model 211 is trained using, for example, a manual for an operation of the medical apparatus. The manual is, for example, an instruction book or data describing a procedure for operating the X-ray diagnosis apparatus 1. The language generative model 211 may also be trained using rules for a sequence of motions of motion axes of the medical apparatus. In this case, the training is performed using, for example, specification data and/or a program defining the sequence of motions of the motion axes of the X-ray diagnosis apparatus 1. The specification data and/or the program may be data not provided to the user, and may be data unique to the apparatus.

An answer output from the language generative model 211 includes a plurality of solutions. That is, the language generative model 211 has been trained to output an answer including a plurality of solutions in response to an input including at least one question. An answer output from the language generative model 211 may include, in addition to a suitable operation method for realizing the user's desire, information irrelevant to the X-ray diagnosis apparatus 1, information that is not an operation method, or solutions such as an operation method that can be carried out only by the X-ray diagnosis apparatus 1 of another manufacturer or other apparatus type, or an operation method that is insufficient to realize the user's desire. Also, the suitable operation method may include a plurality of operations arranged in a suitable order.

The input interface 22 is configured to accept input operations of various kinds from the user, to convert the accepted input operations into electric signals, and to output them to the processing circuitry 25. The user is a healthcare professional such as a technician or a doctor, and is an operator of the X-ray diagnosis apparatus 1. The input interface 22 according to the present embodiment is, for example, connected to an input device such as a microphone, a mouse, a keyboard, a trackball, a switch, a button, a joystick, a touch pad, or a touch panel to which an instruction is input via a touch on an operation screen. The input device connected to the input interface may be an input device provided in another computer connected via a network, etc. A question concerning an operation of the apparatus is input by the user to the input interface 22. The input interface 22 is an example of an input unit.

The display 23 is configured to display various types of information in accordance with an instruction from the processing circuitry 25. For example, the display 23 is configured to display X-ray images generated by an image processing function 252, to be discussed below. Also, the display 23 may be configured to display an input screen for inputting X-ray imaging conditions, X-ray fluoroscopy conditions, SID, etc. The display 23 may be configured, for example, to display a graphical user interface (GUI) for accepting various operations from the user. As the display 23, any display may be suitably employed, for example, a cathode ray tube (CRT) display, a liquid crystal display, an organic EL display, an LED display, or a plasma display. It is to be noted that the X-ray diagnosis apparatus 1 may be configured to not include a display 23, and to display a GUI on an external display, or to display a GUI via a projector, etc. The display 23 is configured to display an answer to the user's question. The display 23 is an example of a display unit.

The communication interface 24 is, for example, an interface for performing communications with a network or an unillustrated external storage device. Data such as X-ray images obtained by the X-ray diagnosis apparatus 1 can be transferred to another device via the communication interface 24 and a network.

The processing circuitry 25 is configured to control motions of the entire X-ray diagnosis apparatus 1 in response to an electric signal of an input operation output from the input interface 22. The processing circuitry 25 is a processor configured to invoke and run programs in the memory 21, thereby implementing a control function 251, an image processing function 252, an input acquiring function 253, an answer acquiring function 254, a determining function 255, and a display control function 256. The processing circuitry 25, which implements the input acquiring function 253, the answer acquiring function 254, the determining function 255, and the display control function 256, realizes the medical information processing apparatus 30 according to the present embodiment.

It is to be noted that the various functions described above are realized by single processing circuitry 25; however, the configuration is not limited thereto. For example, processing circuitry may be configured by a combination of a plurality of independent processors configured to run respective programs to realize the corresponding functions. The above-described functions may be respectively referred to as “control circuitry”, “image processing circuitry”, “question acquiring circuitry”, “answer acquiring circuitry”, “determining circuitry”, and “display control circuitry”, and may be implemented as individual hardware circuits. The above description concerning each of the functions implemented by the processing circuitry 25 is applicable to the embodiments and modifications to be described below.

Hereinafter, the console apparatus 20 will be described as an apparatus configured to implement a plurality of functions with a single console; however, a plurality of functions may be implemented by separate apparatuses. For example, the functions of the processing circuitry 25 may be implemented on different apparatuses in a distributed manner.

The term “processor” used in the above explanation means, for example, circuitry such as a central processing unit (CPU), a graphics processing unit (GPU), an application specific integrated circuit (ASIC), or a programmable logic device (e.g., a simple programmable logic device (SPLD), a complex programmable logic device (CPLD), or a field programmable gate array (FPGA)). If the processor is, for example, a CPU, the processor reads and runs programs stored in the storage circuitry to realize the respective functions. On the other hand, if the processor is, for example, an ASIC, instead of storing a program in a storage circuit, a corresponding function is directly incorporated into a circuit of the processor as a logic circuit. Each processor of the present embodiment is not necessarily configured as a single circuit, and a plurality of independent circuits may be combined into a single processor to realize the respective functions. In addition, a plurality of structural elements shown in FIG. 1 may be integrated into a single processor to realize the respective functions. The above description of the “processor” is applicable to the subsequent embodiments and modifications.

The processing circuitry 25 is configured, with the control function 251, to control the entire X-ray diagnosis apparatus 1 in a unified manner. Specifically, the processing circuitry 25 is configured to read control programs stored in the memory 21 and expand them on a memory, and control the respective elements of the X-ray diagnosis apparatus 1 in accordance with the expanded control programs. The processing circuitry 25, which realizes the control function 251, is an example of a system control unit.

The processing circuitry 25 is configured, with the control function 251, to control the elements of the X-ray diagnosis apparatus 1 using command signals and various initial setting conditions input by the user via the input interface 22. For example, the processing circuitry 25 is configured, with the control function 251, to control the driving unit 13 and the top plate driving unit using information concerning driving of the movement support mechanism 9 and the rotation support mechanism 11 input via the input interface 22. For example, the processing circuitry 25 is configured, with the control function 251, to perform control of rotation and sliding of the top plate 14 and movement of the imaging system.

The processing circuitry 25 is further configured, with the control function 251, to control the X-ray radiation range using, for example, information concerning driving of the radiation range limiter 5 input from the input interface 22.

The processing circuitry 25 is further configured, with the control function 251, to read information on various initial setting conditions and controls imaging conditions such as a tube voltage, a tube current, and an exposure time of the high-voltage generator. The imaging conditions may further include a product (mAs) of the tube current and the exposure time.

The processing circuitry 25 is configured, with the image processing function 252, to generate an X-ray image of a subject based on an output from the X-ray detector 7. For example, the processing circuitry 25 is configured, with the image processing function 252, to collect fluoroscopic images and radiographic images based on an output from the X-ray detector 7, to perform various kinds of image processing for display, and to cause the display 23 to display X-ray images of the subject such as fluoroscopic images and radiographic images. The processing circuitry 25 is further configured, with the image processing function 252, to generate fluoroscopic moving images and a current X-ray still image, and to cause the display 23 to display the generated images. The processing circuitry 25 may be further configured, with the image processing function 252, to read past examination images corresponding to the current X-ray image from the memory 21, and to cause the display 23 to display the current X-ray image and the past examination images in parallel. The processing circuitry 25, which realizes the image processing function 252, is an example of an image generating unit and an image processing unit.

The processing circuitry 25 is configured, with the input acquiring function 253, to acquire a question concerning an operation of the medical apparatus. At this time, the processing circuitry 25 is configured to acquire a question input by the user using the input interface 22. A question is, for example, a question sentence input to the language generative model 211 to make an inquiry about the operation of the X-ray diagnosis apparatus 1. A question may be configured of a single word, or may be configured of a plurality of sentences. What is acquired by the processing circuitry 25 is not limited to a question, and may be an order concerning an operation of the X-ray diagnosis apparatus 1. An order is, for example, an imperative sentence such as “Output a method for switching the apparatus to the upright mode”. The processing circuitry 25, which realizes the input acquiring function 253, is an example of an acquiring unit configured to acquire a user input concerning an operation of the medical apparatus.

The processing circuitry 25 is configured, with the answer acquiring function 254, to input the acquired question to the language generative model 211, to cause the language generative model 211 to output an answer to the question, and to acquire the answer output from the language generative model 211. The answer output from the language generative model 211 includes one or more solutions. The processing circuitry 25, which realizes the answer acquiring function 254, is an example of an answer acquiring unit.

The processing circuitry 25 is configured, with the determining function 255, to determine a feasibility of each of the solutions output from the language generative model 211 in a local apparatus. The feasibility is an index indicating a degree to which each solution can be implemented in the local apparatus. The “local apparatus” refers to an X-ray diagnosis apparatus 1 on which the processing circuitry 25 is mounted, and is identified by the purpose, the manufacturer name, the apparatus type, and the model number of the X-ray diagnosis apparatus. It is to be noted that an apparatus that matches the X-ray diagnosis apparatus in terms of at least one of the purpose, the manufacturer name, the apparatus type, and the model number may be treated as the “local apparatus”. For example, the more operations implementable in the local apparatus a solution includes, the higher the feasibility of the solution becomes. Also, an index indicating a degree to which the user's desire is realized through implementation of the solution in the local apparatus may be used as the feasibility. The feasibility may be a numerical value, a result of classification into a plurality of items set in advance, or information indicating whether or not it is implementable in the local apparatus. The processing circuitry 25, which realizes the determining function 255, is an example of a determining unit.

For determination of the feasibility of a solution, a simulation of a motion of the local apparatus, for example, can be used. In this case, the processing circuitry 25 simulates a motion of the local apparatus operated in accordance with the solution, and determines a feasibility in accordance with a result of the simulation. The simulation may be performed using, for example, an interference control program or digital twin technology. Moreover, by determining whether or not a result of a simulation satisfies predetermined conditions set in advance, it may be determined that a feasibility of a solution that satisfies the conditions is high and that a feasibility of a solution that does not satisfy the conditions is low. Furthermore, by performing determinations with respect to a plurality of determination items using a result of a simulation and performing a weighted addition of the results of the determinations, a feasibility evaluation value may be calculated. Items that can be used for the determination items include, for example, whether or not the solution includes contents related to the medical apparatus, whether or not the solution is realizable in the local apparatus, whether or not the solution is applicable to the current situation of the local apparatus, and whether or not the solution fulfills the user's desire.

The processing circuitry 25 is configured, with the display control function 256, to cause the display 23 to display information corresponding to an output from the language generative model 211 in accordance with the result of the determination of the feasibility. For example, the processing circuitry 25 is configured to extract, from among a plurality of solutions output from the language generative model 211, one or more solutions with a feasibility higher than a predetermined value, and cause only the extracted solutions to be displayed. Moreover, the processing circuitry 25 may be configured to cause the plurality of solutions output from the language generative model 211 to be displayed in ascending order of feasibility. Furthermore, the processing circuitry 25 may be configured to process and display the solutions output from the language generative model 211. The processing circuitry 25, which realizes the display control function 256, is an example of a display control unit.

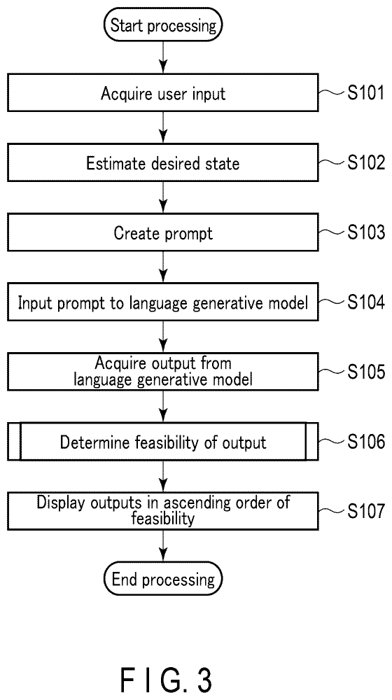

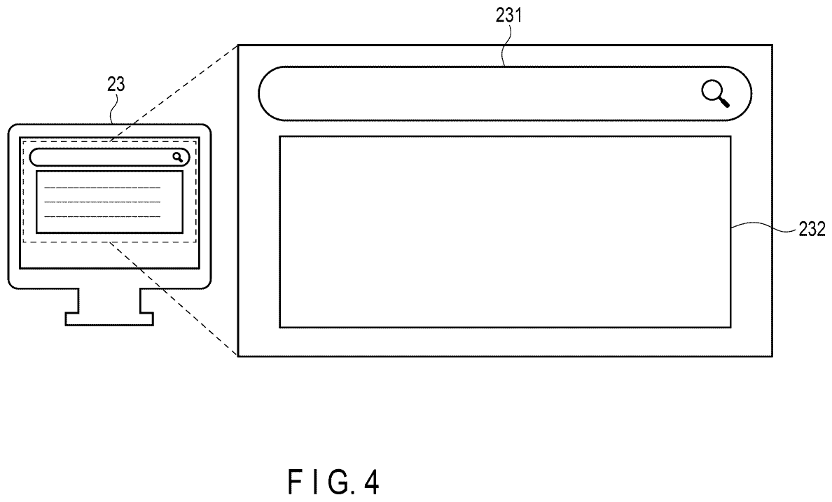

Next, motions of the X-ray diagnosis apparatus 1 according to the present embodiment will be described. FIG. 3 is a flowchart showing an example of a procedure for an operation assisting process performed by the processing circuitry 25. The operation assisting process is a process of assisting a user's operation in the case where the user does not know how to operate the X-ray diagnosis apparatus 1 by presenting a suitable operation method in the form of an answer to a question input from the user, using a language generative model. FIG. 4 shows an example of a display screen (hereinafter referred to as an “operation assist screen”) displayed on the display 23 in an operation assisting process. As shown in FIG. 4, the operation assist screen includes a question input unit 231 and an answer display unit 232. The question input unit 231 is configured to receive a question from the user as an input. The answer display unit 232 displays, according to the feasibility, one or more solutions to the question generated using an output from the language generative model 211.

It is to be noted that the processing procedure to be described below is merely an example, and that the processing can be changed to the extent possible. Omission, replacement, or addition of a step in the processing procedure to be described below can be suitably made, in accordance with an actual situation where the present embodiment is realized.

### Operation Assisting Process

### Step S101

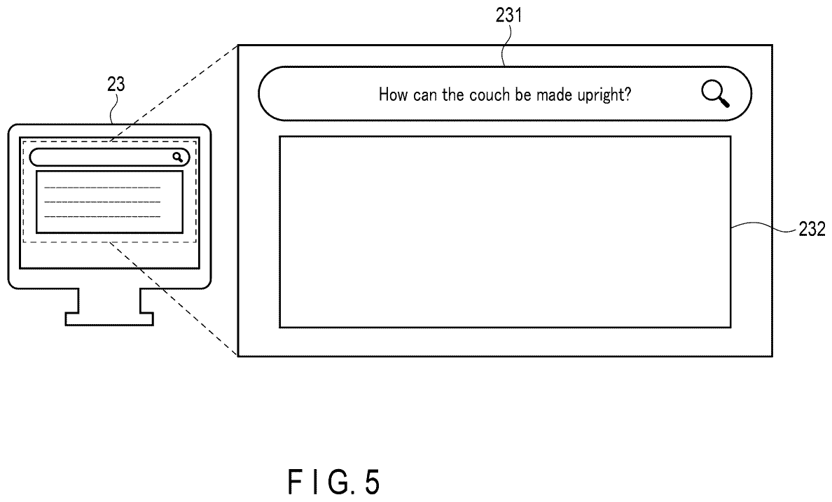

In an operation assisting process, the processing circuitry 25 acquires, with the input acquiring function 253, a question input to the question input unit 231 as a user input, based on an operation signal acquired from the input interface 22. FIG. 5 shows a case where a question “How can the couch be made upright?” is input to the question input unit 231.

### Step S102

Subsequently, with the determining function 255, the processing circuitry 25 performs language analysis of the input question using natural language processing, and estimates an after-operation state of the local apparatus which the user desires to achieve through an operation. The after-operation state of the local apparatus which the user desires to achieve will be hereinafter referred to as a “desired state”. The desired state is an example of a target state to be attained by the user, and may also be referred to as a “final state desired by the user”. The desired state is estimated by, for example, separating a question by clauses using natural language processing, and converting words in each clause into numerical values or symbols using general definitions of the words such as “couch” and “upright”. Alternatively, the desired state may be estimated by employing apparatus-specific conversion rules in which correspondences between the words and the numerical values or symbols are set in advance. It is assumed herein that, in response to the question “How can the couch be made upright?”, a state of the couch at a rotation angle of 80° or greater and 90° or less is estimated as the desired state.

### Step S103

Subsequently, with the answer acquiring function 254, the processing circuitry 25 generates a prompt to be input to the language generative model 211 based on the input question. Specifically, the processing circuitry 25 converts a desired state generated based on the question, and creates a prompt to give an instruction or order to the language generative model 211.

### Step S104

Subsequently, with the answer acquiring function 254, the processing circuitry 25 inputs the created prompt to the language generative model 211, and causes the language generative model 211 to output one or more solutions to the question.

### Step S105

Subsequently, with the answer acquiring function 254, the processing circuitry 25 acquires the one or more solutions output from the language generative model 211. It is assumed herein that a plurality of solutions have been output in response to the question “How can the couch be made upright?”.

### Step S106

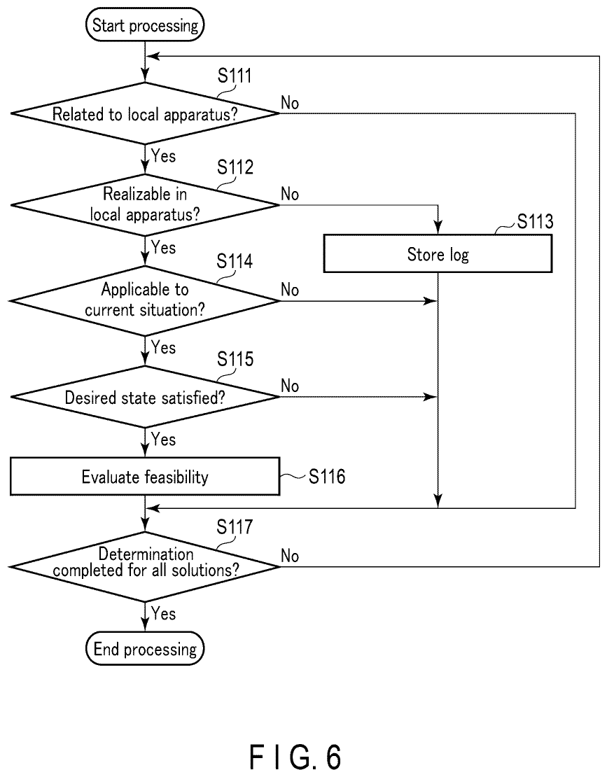

Subsequently, with the determining function 255, the processing circuitry 25 performs a determination process of determining a feasibility of implementing each of the solutions output from the language generative model 211 in the local apparatus. FIG. 6 is a flowchart showing an example of a procedure for the determination process performed by the processing circuitry 25 with the determining function 255.

### Determination Process

### Step S111

In a determination process, the processing circuitry 25 determines, at step S111, whether or not each of the solutions output from the language generative model 211 includes contents related to an operation of the local apparatus. At this time, the processing circuitry 25 performs natural language processing on each of the solutions, and extracts keywords therefrom. After that, the processing circuitry 25 calculates a similarity score between the extracted keywords and a manual of the local apparatus. If the similarity score between the keywords and the manual is high (step S111—Yes), the processing circuitry 25 determines that the solution includes contents related to an operation of the local apparatus. On the other hand, if the similarity score between the keywords and the manual is low (step S111—No), the processing circuitry 25 determines that the contents of the solution are not related to an operation of the local apparatus, and excludes the solution from candidates to be displayed. The solution excluded from the candidates to be displayed will not be presented to the user, and will not be subjected to the feasibility evaluation.

### Step S112

Subsequently, the processing circuitry 25 determines whether or not each solution is realizable in the local apparatus. At this time, the processing circuitry 25 simulates a case where the solution is implemented in the local apparatus, and determines that the solution is unrealizable in the local apparatus if an error to be handled or a potential bug has occurred in a result of the simulation. For example, the processing circuitry 25 implements, in accordance with the solution, the interference control program stored in advance in the memory 21, and checks whether or not a default error or an unintended bug would occur if the X-ray diagnosis apparatus 1 were operated in accordance with the solution. An error occurs in the case where, for example, a movable part to be operated by an operation method included in the solution cannot be identified due to the local apparatus including more movable parts than a general X-ray diagnosis apparatus, or a movable part to be operated interferes with another movable part not installed in a general apparatus type. If an error or a bug does not occur, the processing circuitry 25 determines that the solution is realizable in the local apparatus (step S112—Yes). On the other hand, if an error or a bug has occurred, the processing circuitry 25 determines that the solution is unrealizable in the local apparatus (step S112—No), and excludes the solution from the candidates to be displayed. The solution excluded from the candidates to be displayed will not be presented to the user, and will not be subjected to the feasibility evaluation.

### Step S113

If an error or a bug has occurred at step S112 (step S112—No), the processing circuitry 25 stores contents of the error or the bug as a log in the memory 21. The error or the bug stored as the log can be used for training of the language generative model 211 and/or maintenance of the X-ray diagnosis apparatus 1.

### Step S114

Subsequently, the processing circuitry 25 determines whether or not the solution takes the current situation of the local apparatus into account. At this time, the processing circuitry 25 determines, based on situation information concerning the current situation of the local apparatus, whether or not the solution is applicable to the current situation of the local apparatus. The situation information includes a current position of each axis of the X-ray diagnosis apparatus 1, an operation situation of the X-ray diagnosis apparatus 1, a motion situation of the X-ray diagnosis apparatus 1, a current arrangement situation of people and/or objects in an examination room, examination information of a patient to be inspected, and the like. The current arrangement situation of people and/or objects in the examination room is acquired by, for example, analyzing camera images captured within the examination room. The situation information is stored in advance in, for example, the memory 21. The current arrangement situation of people and/or objects in the examination room may be acquired by analyzing speech data acquired from a microphone installed in the examination room.

The situation information of the X-ray diagnosis apparatus 1 may be acquired using digital twin technology. In this case, a digital twin can be created using, for example, an operational history of the local apparatus. The operational history includes, for example, a motion history of each axis, an operation history, a motion situation, etc. The motion situation includes, for example, the number of hours of use, the number of days of use, the frequency of use, etc. The motion history of each axis is acquired by, for example, an encoder, etc. using a sensor value. By creating a digital twin using the operational history of the local apparatus, the current situation of the local apparatus is reflected on the digital twin. If the situation information is acquired using digital twin technology, information related to a state of the interior of the X-ray diagnosis apparatus 1 can be employed, thus making it possible to acquire a large amount of situation information with high accuracy, compared to the case of acquiring situation information using camera images.

For example, the processing circuitry 25 determines, using the result of the simulation performed at step S112, whether or not there would be a danger of collision of a movable part of the X-ray diagnosis apparatus 1 with a person or object in the examination room if the local apparatus were operated in accordance with the solution, and determines, if there would be a danger of collision, that the solution is inapplicable to the current situation of the local apparatus. Alternatively, the processing circuitry 25 may calculate, based on the current position of each axis of the X-ray diagnosis apparatus 1, the number of hours and the number of steps required for executing the operation of the solution in the local apparatus, and determine, if the calculated number of hours and/or number of steps exceeds predetermined values, that the solution is inapplicable to the current situation of the local apparatus. Alternatively, the processing circuitry 25 may search for an operation that is burdensome to a patient from a plurality of operations included in the solution based on examination information of the patient, and determine, if an operation that is burdensome to the patient is included, that the solution is inapplicable to the current situation of the local apparatus.

If the solution is determined to be applicable to the current situation of the local apparatus, the processing circuitry 25 determines that the solution takes the current situation of the local apparatus into account (step S114—Yes). On the other hand, if the solution is determined to be inapplicable to the current situation of the local apparatus, the processing circuitry 25 determines that the solution does not take the current situation of the local apparatus into account (step S114—No), and excludes the solution from the candidates to be displayed. The solution excluded from the candidates to be displayed will not be presented to the user, and will not be subjected to the feasibility evaluation.

### Step S115

Subsequently, the processing circuitry 25 determines whether or not a state (hereinafter referred to as an “after-operation state”) after implementation of the solution proposed by the language generative model 211 satisfies conditions for the desired state. At this time, the processing circuitry 25 predicts an after-operation state, and determines the feasibility of the solution by comparing the after-operation state with the desired state predicted at step S102. The after-operation state may be predicted using the result of the simulation performed at step S112, or by performing a new simulation related to a desired state. If, for example, the desired state refers to a state of the couch at a rotation angle of 80° or greater and 90° or less and the after-operation state refers to a state of the couch at a rotation angle of approximately 89°, the processing circuitry 25 determines that the after-operation state is a solution that satisfies the conditions for the desired state (step S115—Yes). On the other hand, if the after-operation state does not satisfy the conditions for the desired state (step S115—No), the processing circuitry 25 excludes the solution from the candidates to be displayed. The solution excluded from the candidates to be displayed will not be presented to the user, and will not be subjected to the feasibility evaluation.

### Step S116

Subsequently, the processing circuitry 25 evaluates the feasibility of the solution based on the contents of the determination at steps S112 to S115. For example, the processing circuitry 25 converts the contents of the determinations performed at steps S112 to S115 into numerical values according to default rules, and obtains a feasibility evaluation value by adding the numerical values. For example, if it has become clear from the examination information that CT imaging is to be performed on a patient sitting in a wheelchair, it is preferable to decrease the feasibility evaluation value, since assistance will be needed for performing upright imaging.

### Step S117

Subsequently, the processing circuitry 25 determines whether or not the feasibility determination has been completed for all of the solutions proposed by the language generative model 211. If there is a solution remaining for which the feasibility determination has not been completed (step S117—No), the processing returns to step S111.

The processing circuitry 25 repeatedly executes steps S111 to S116 for each of the solutions, and obtains an evaluation value for all of the solutions that are not excluded from the candidates to be displayed. If the above-described processing has been performed for all of the solutions (step S117—Yes), the processing circuitry 25 ends the determination process at step S106.

### Step S107

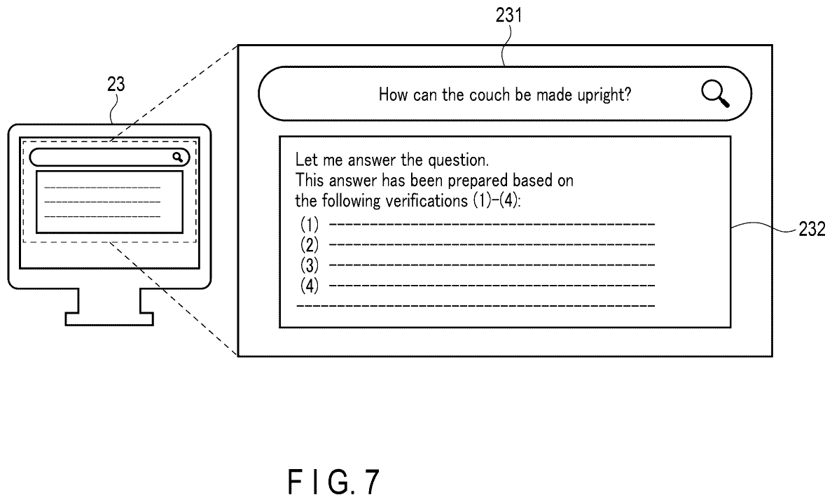

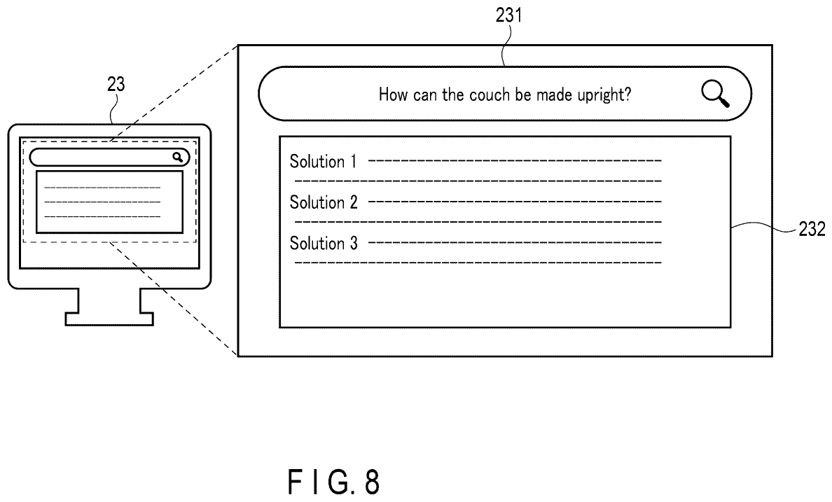

After the determination process has been completed, with the display control function 256, the processing circuitry 25 causes the display 23 to display the remaining solutions as the candidates to be displayed in the form of answers to the question. At this time, the processing circuitry 25 causes the answer display unit 232 on the operation assist screen to display the solutions. FIGS. 7 and 8 are diagrams each showing an example of answers displayed on the answer display unit 232. FIG. 8 shows a display example in the case where the contents displayed on the answer display unit 232 shown in FIG. 7 are scrolled down. In FIG. 7, the answer display unit 232 displays what specific verifications have been performed for the feasibility determination at step S107. For example, the answer display unit 232 displays results of the feasibility determinations at steps S111 to S115 and information used for the determinations. The answer display unit 232 displays, for example, the type, the figure number, and the page number of the manual referred to in performing a simulation. Alternatively, if there is a risk of contact with a surrounding installation, a photograph of the installation may be displayed on the answer display unit 232.

The answer display unit 232 shown in FIG. 8 displays the remaining solutions as the candidates to be displayed in ascending order of evaluation values. At this time, the answer display unit 232 does not display the solutions excluded from the candidates to be displayed in the processing at steps S111 to S115.

In the following, advantageous effects of the X-ray diagnosis apparatus 1 according to the present embodiment will be described.

The X-ray diagnosis apparatus 1 comprising medical information processing apparatus according to the present embodiment is configured to: acquire, as a user input, a question concerning an operation of the apparatus; input the acquired question concerning the operation of the apparatus to a language generative model 211 having trained to, upon accepting, as an input, a question concerning the operation of the apparatus, output an answer to the accepted question and acquire an output from the language generative model 211; determine a feasibility of the output acquired from the language generative model 211 in the local apparatus; and cause the display 23 to display information corresponding to the output in accordance with the feasibility.

With the above-described configuration, the X-ray diagnosis apparatus 1 according to the present embodiment has a function of answering a question concerning a user's operation by extracting one or more solutions using language generative AI such as ChatGPT (registered trademark). Moreover, the X-ray diagnosis apparatus 1 according to the present embodiment is capable of proposing solutions for which validity has been confirmed by verifying feasibilities of the solutions extracted by the language generative AI in the local apparatus to confirm validity of the solutions prior to offering the solutions to the user as answers. It is thereby possible to precisely support an operation desired by the user, allowing the user to efficiently perform the operation. Also, by using the language generative AI, solutions each including a plurality of operations can be listed in a suitable order and presented to the user.

It is possible, for example, to confirm that each of the solutions proposed by the language generative AI includes contents related to an operation of the local apparatus, is not unrealizable in the local apparatus, takes the current situation of the local apparatus into consideration, and fulfills a final goal desired by the user, thereby evaluating the feasibility based on the results of the confirmation. The feasibility of each item can be confirmed using, for example, a result of simulating a motion in the case of implementing each of the solutions in the local apparatus. In the present embodiment, determinations have been made with respect to four determination items in the processing from steps S111 to S115; however, the feasibility determination should be performed with respect to at least one of the above-noted items, and determination with respect to any one of the items may be omitted.

Moreover, in the present embodiment, since solutions excluded from the candidates to be displayed are not displayed as the answer, the user can confirm only solutions that are implementable in the local apparatus by checking only the remaining solutions as the candidates to be displayed. Furthermore, in the present embodiment, since solutions are displayed in ascending order of feasibility evaluation value, the user can preferentially confirm suitable solutions by checking the displayed solutions from top to bottom.

### Modifications

In the present embodiment, the feasibility evaluation value is calculated only for the remaining solutions as the candidates to be displayed; however, the feasibility evaluation value may be calculated for all of the solutions, and only solutions with an evaluation value equal to or greater than a predetermined value may be displayed. Moreover, feasibility evaluation values of all the solutions may be calculated, and all the solutions may be displayed in ascending order of evaluation value.

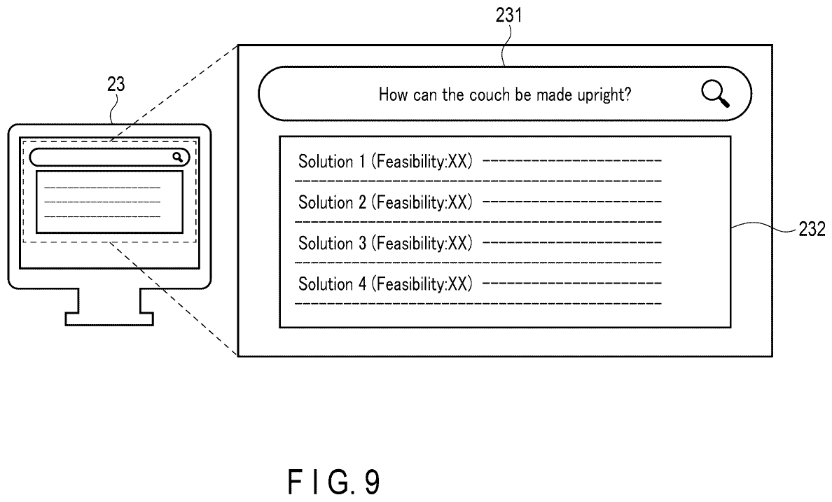

As shown in FIG. 9, in addition to the solutions, the feasibility evaluation values of the respective solutions may be displayed. In this case, with the display control function 256, the processing circuitry 25 causes the display 23 to display the feasibilities of the respective solutions.

Furthermore, situation information used for determining the feasibility, information concerning a risk detected based on the situation information, and the like may be displayed together with the solutions. If, for example, situation information is displayed for each of the solutions, the processing circuitry 25 may display, with the display control function 256, the time and the number of steps required to implement each solution in the local apparatus together with the solutions. Alternatively, an operation that is burdensome to the patient extracted from a plurality of operations included in each of the solutions may be displayed together with the solutions, to prompt the user's attention and action.

In the above-described embodiment, the medical information processing apparatus 30 mounted on the X-ray diagnosis apparatus 1 has been described; however, the medical information processing apparatus is applicable to an apparatus equipped with a function of answering a question concerning a method for operating the apparatus. The medical information processing apparatus may be mounted on a medical apparatus other than a medical diagnosis apparatus, or may be mounted on an apparatus other than a medical apparatus.

For example, a medical information processing apparatus may be mounted on a general PC terminal or a smartphone terminal equipped with a function of answering a question concerning an operation using language generative AI. In this case, a plurality of solutions to a question concerning a method for operating the terminal are acquired from language generative AI, feasibilities of the respective solutions are determined, and the solutions are presented to the user according to the feasibility. For the feasibility determination, online manuals or a support page of the sales company of the local apparatus, for example, may be used. In this case, a link to or an image of online information referred to for the feasibility determination may be displayed, in addition to the solutions. Moreover, a motion in the case of implementing each of the solutions in the local apparatus may be simulated using digital twin technology to determine its feasibility. In this case, a simulation video based on digital twin technology may be displayed, together with the solutions. For example, a video showing how a cursor moves and how the screen transitions if an operation included in a solution is performed should be generated as the simulation video.

According to at least one embodiment described above, it is possible to efficiently support the user in terms of the operation of the medical apparatus.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus, comprising processing circuitry configured to:
acquire a user input concerning an operation of a medical apparatus;
input the acquired user input concerning the operation of the medical apparatus into a trained model, and acquire an output from the trained model, the trained model having learned to, upon accepting, as an input, a user input, output an answer to the accepted user input;
determine a feasibility of the acquired output in a local apparatus; and
cause a display to display information corresponding to the output in accordance with the feasibility.

2. The medical information processing apparatus according to claim 1, wherein
the processing circuitry is configured to simulate a case where the output is applied to the local apparatus, and to determine the feasibility of the output based on a result of the simulating.

3. The medical information processing apparatus according to claim 2, wherein
the processing circuitry is configured to determine that the feasibility of the output is high if the result of the simulating satisfies a predetermined condition.

4. The medical information processing apparatus according to claim 3, wherein
the processing circuitry is configured to determine that the feasibility of the output is low if the result of the simulating includes an error or a bug.

5. The medical information processing apparatus according to claim 4, wherein
the processing circuitry is configured, if the result of the simulating includes an error or a bug, to store the error or the bug as a log.

6. The medical information processing apparatus according to claim 1, wherein
the processing circuitry is configured to determine the feasibility of the output based on situation information including information, an operation situation, and a motion situation of the local apparatus, or position information of a peripheral object.

7. The medical information processing apparatus according to claim 6, wherein
the processing circuitry is configured to simulate a case where the output is applied to the local apparatus based on the situation information, and to determine the feasibility of the output based on a result of the simulating.

8. The medical information processing apparatus according to claim 6, wherein
the processing circuitry is configured to cause the display to display the situation information together with the output.

9. The medical information processing apparatus according to claim 1, wherein
the processing circuitry is configured to determine whether or not the output is information concerning the operation of the local apparatus, and to determine the feasibility of the output based on a result of the determining.

10. The medical information processing apparatus according to claim 1, wherein
the processing circuitry is configured to determine the feasibility of the output by inferring, based on the user input, a target state to be attained by a user, inferring an after-operation state in which the output has been implemented, and comparing the after-operation state with the target state.

11. The medical information processing apparatus according to claim 1, wherein
the trained model is configured to output an answer including a plurality of solutions to an input including a single user input, and
the processing circuitry is configured to cause, from among the plurality of solutions, only a solution with a high feasibility to be displayed.

12. The medical information processing apparatus according to claim 1, wherein
the trained model is configured to output an answer including a plurality of solutions to an input including a single user input, and
the processing circuitry is configured to cause the plurality of solutions to be displayed in ascending order of feasibility.

13. The medical information processing apparatus according to claim 1, wherein
the trained model is configured to output an answer including a plurality of solutions to an input including a single user input, and
the processing circuitry is configured to cause the plurality of solutions and feasibilities of the plurality of solutions to be displayed.

14. A medical diagnosis apparatus, comprising:
the medical information processing apparatus according to claim 1;
a storage unit configured to store the trained model;
processing circuitry configured to generate a medical image of a subject;
an input unit configured to receive the user input as an input; and
the display configured to display information corresponding to the output.

15. The medical diagnosis apparatus according to claim 14, wherein
the medical diagnosis apparatus is one of an X-ray diagnosis apparatus, an X-ray CT apparatus, an MRI apparatus, or an ultrasound diagnosis apparatus.

16. The medical diagnosis apparatus according to claim 15, wherein
the X-ray diagnosis apparatus is an X-ray TV couch apparatus.

17. The medical information processing apparatus according to claim 1, wherein
the processing circuitry is configured to determine the feasibility of the output using a digital twin.

18. The medical information processing apparatus according to claim 17, wherein
the processing circuitry is configured to cause the display to display a result of the digital twin.

19. A medical information processing method using the medical information processing apparatus according to claim 1, comprising:
acquiring the user input;
inputting the acquired user input into the trained model and acquire an output from the trained model;
determining the feasibility of the acquired output; and
causing the display to display information corresponding to an output with a high feasibility.
